(19) United States
(12) Patent Application Publication
(10) Pub. No.: US 2022/0162247 A1
(43) Pub. Date: May 26, 2022

(54) Production And Use Of Metal Organic Frameworks

(71) Applicant: ExxonMobil Research and Engineering Company, Annandale, NJ (US)

(72) Inventors: Joseph M. Falkowski, Hampton, NJ (US); Pavel Kortunov, Flemington, NJ (US); Yogesh V. Joshi, Bridgewater, NJ (US); Gerardo J. Majano, Basking Ridge, NJ (US)

(21) Appl. No.: 17/310,615

(22) PCT Filed: Apr. 1, 2020
(86) PCT No.: PCT/US2020/026211

§ 371 Date: Aug. 13, 2021

Related U.S. Application Data
(60) Provisional application No. 62/832,919, filed on Apr. 12, 2019.

(51) Int. Cl. C07F 15/02; C01B 37/00

(52) U.S. Cl. CPC C07F 15/02; C01B 37/00

(57) ABSTRACT

A process for producing a bimetallic, terephthalate metal organic framework (MOF) having a flexible structure and comprising aluminum and iron cations, comprises contacting a water-soluble aluminum salt, a chelated iron compound and 1,4-benzenedicarboxylic acid or a salt thereof with a fluoride-free mixture of water and a polar organic solvent at a reaction temperature of less than 200° C. to produce a solid reaction product comprising the MOF.

### FIELD

The present disclosure relates to the production and use of metal organic frameworks (MOFs), and in particular terephthalate MOFs having a flexible structure, such as MIL-53 and MOFs similar to MIL-53.

### BACKGROUND

Metal-organic frameworks (MOFs) are porous crystalline materials prepared by the self-assembly of metal ions and organic ligands. MOFs can have large pore volumes and apparent surface areas as high as 8,000 m²/g. MOFs combine a structural and chemical diversity that make them attractive for many potential applications, including gas storage, gas separation and purification, sensing, catalysis and drug delivery. The most striking advantage of MOFs over more traditional porous materials is the possibility to tune the host/guest interaction by choosing the appropriate building blocks, i.e. the metal ions and organic ligands, from which the MOF is formed. In addition, compared to purely inorganic zeotypes, MOFs can show unique structural features, one striking example of which is large structural flexibility, where reversible expansion and contraction may occur in response to change in temperature or introduction and removal of guest molecules.

One MOF material of particular interest is MIL-53. This material has the general chemical composition M^{III}(BDC)(OH) and consists of one-dimensional (1-D) chains of trans linked metal-oxide octahedra cross-linked to one another by 1,4-benzenedicarboxylate (BDC) dianions. In the simplest form of this material, the metal is trivalent and in an octahedral environment, coordinated to four oxygen atoms from 1,4-benzenedicarboxylates and two from the trans bridging μ2-hydroxyl groups. The interconnectivity of the 1-D metal-oxide chains with the BDC linkers leads to a structure with 1-D, diamond-shaped channels running parallel to the hydroxide chains. These channels are typically occupied by solvent and/or unreacted 1,4-benzenedicarboxylic acid and can be evacuated using elevated temperatures or reduced pressure. The flexibility of the MIL-53 structure has been well documented: with temperature, pressure, or the addition of guest molecules, the framework may undergo a dramatic expansion, involving displacement of atoms by several Angstroms while the topology of the structure is maintained. The flexibility of the MIL-53 structure is also dependent on the nature of the metal and the organic linking anion. To date, attempts to modify the flexibility and adsorption properties of MIL-53 materials have primarily focused on modifying the organic linkers with varying functional groups. However, a simpler and more intuitive method of modifying MIL-53 behavior would be the synthesis of bimetallic MIL-53 materials, particularly with metals having antagonistic behavior to the flexibility of the structure. For example, the chromium and aluminum materials convert to a fully open, or “LP” (large pore) structure, upon heating, with a large increase in pore volume, whereas the iron analogue undergoes a slight contraction of its structure. Even upon heating further, MIL-53(Fe) only slightly expands, essentially remaining in the “NP” (narrow pore) structure.

One bimetallic approach to tuning the adsorption properties of MIL-53 is described by Breeze, M. I.; Clet, G.; Campo, B. C.; Vimont, A.; Daturi, M.; Greneche, J-M.; Dent, A. J.; Millange, F. and Walton, F. I. in an article entitled “Isomorphous Substitution in a Flexible Metal-Organic Framework: Mixed-Metal, Mixed-Valent MIL-53 Type Materials”, Inorg. Chem. 2013, 52, 8171-8172. In this approach, mixed iron-vanadium analogues of MIL-53 were synthesized by heating the metal chlorides together with 1,4-benzenedicarboxylic acid in a mixture of N,N′-dimethylformamide, water and hydrofluoric acid at a temperature of 170 to 200° C. for up to 3 days. However, the ability to tune the MIL-53 composition by this approach was limited, in that the highest vanadium content obtainable was 50%. According to the authors, attempts to increase the vanadium content above this value led “to the formation of the known V(III) phase MIL-68(V)”.

A similar direct synthesis approach to the production of MIL-53(Cr—Fe) is described by Nouar, F.; Devic, T.; Guillou, N.; Gibson, E.; Clet, G.; Daturi, M.; Vimont, A.; Greneche J-M.; Breeze, M. I.; Walton, R. I.; Llewellyn, P. L.; and Serre, C. in “Tuning the breathing behaviour of MIL-53 by cation mixing”, Chem. Commun. 2012, 48, 10237-10239. This approach involves heating a stoichiometric mixture of chromium nitrate, iron powder, hydrofluoric acid and terephthalic acid in water at 453 K for 4 days. However, not only do the authors fail to demonstrate an ability to precisely tune the cation content, and as such the adsorption properties, through their synthetic technique, but also this synthesis requires harsh mineralizing conditions (high temperature, hydrofluoric acid) to allow the incorporation of iron into the chromium-based material.

An alternative approach involving solid-solid cation exchange between, for example, MIL-53(Al) and MIL-53(Fe) is described by Kim, M.; Cahill, J. F; Fei, H.; Prather, K. A.; and Cohen, S. M. in an article entitled “Postsynthetic Ligand and Cation Exchange in Robust Metal-Organic Frameworks”, J. Am. Chem. Soc. 2012, 134, 18082-18088. This mechanism, besides being indirect, resulted in materials with a large range in metal composition. In their experiments, the authors observed that >60% of the material remained unchanged (i.e., still contained 100% Fe or 100% Al). Additionally, the authors did not elaborate what concentrations of iron and aluminum they were able to obtain. However based on the results presented, it is expected that an extremely broad distribution is only obtainable.

There is therefore a need for new methods of producing bimetallic forms of MIL-53 and similar flexible MOFs which provide the ability to tune the metal ratios over a wide degree and with fine control and yet can be conducted under mild conditions without the use of caustic/toxic solvents.

### SUMMARY

According to the present invention, it has now been found that Al/Fe-containing terephthalate MOFs having a flexible structure consistent with MIL-53 can be produced from a fluoride-free mixed solvent system under relatively mild conditions. The process allows tuning of the Al:Fe ratio over a wide range and with fine control allowing for unique and predictable adsorption phenomena to be accessed.

Thus, in one aspect, there is provided a process for producing a terephthalate metal organic framework (MOF) having a flexible structure and comprising aluminum and iron cations, the process comprising:

(a) providing a fluoride-free mixture of water and a polar organic solvent;

(b) contacting a water-soluble aluminum salt, a chelated iron compound and 1,4-benzenedicarboxylic acid or a derivative or a salt thereof with the mixture at a reaction temperature of less than 200° C. to produce a solid reaction product comprising the MOF; and

(c) recovering the MOF from the mixture,

wherein the recovered MOF product, when subjected to X-ray diffraction analysis at 200° C. under a flowing atmosphere of N₂, exhibits a pattern including at least the characteristic lines listed in Table 1:

TABLE 1

| Interplanar | Relative Intensity |
|---|---|
| d-Spacing (Å) | (100 × I/Io) |
| 10.0 ± 0.2 | s-vs |
| 6.5 ± 0.2 | w-s |
| 4.9 ± 0.2 | w-m |
| 3.34 ± 0.2 | vw |

In another aspect, there is provided a process for producing a terephthalate metal organic framework (MOF) having a flexible structure and comprising aluminum and iron cations, the process comprising:

(a) providing a fluoride-free mixture of water and a polar organic solvent;

(b) contacting a water-soluble aluminum salt, a chelated iron compound and 1,4-benzenedicarboxylic acid or a derivative or a salt thereof with the mixture at a reaction temperature of less than 200° C. to produce a solid reaction product comprising the MOF; and

(c) recovering the MOF from the mixture,

wherein the recovered MOF product, when analyzed by methane adsorption, exhibits an inflection in the adsorption isotherm at pressures below 8 bar (the pressure of the inflection in the adsorption isotherm for pure MIL-53 (Fe)).

In further aspects, the invention resides in Al/Fe-containing terephthalate MOFs having a flexible structure produced by the processes described herein and use of the resultant MOFs in the adsorption of methane.

### BRIEF DESCRIPTION OF THE DRAWINGS

FIG. 1 shows the X-ray diffraction patterns of the MOF products of Examples 1 to 3 (containing different amounts of aluminum and iron) conducted at 200° C. (top) and 30° C. (bottom) respectively.

FIG. 2 compares the gravimetric methane adsorption isotherms conducted at 30° C. on the MOF products of Examples 1 to 3 with those of samples containing 100% aluminum and 100% iron.

FIG. 3 shows the volumetric methane adsorption isotherm conducted at 30° C. of the MOF product of Example 1 (containing about 50 mol. % Al based on the total metal content as measured by EDX).

### DETAILED DESCRIPTION OF THE EMBODIMENTS

The present disclosure provides a new and advantageous process for producing a terephthalate metal organic framework (MOF) having a flexible structure and comprising aluminum and iron cations. The present process comprises providing a fluoride-free mixture of water and a polar organic solvent and then contacting the mixture with water-soluble aluminum salt, a chelated iron compound and 1,4-benzenedicarboxylic acid or a salt thereof at a reaction temperature of less than 200° C. to produce a solid reaction product comprising an Al/Fe-containing MOF having a flexible structure similar to that of MIL-53. The MOF can then be recovered from the mixture.

Polar organic solvents, including solvents which are miscible with water and those that are immiscible with water, can be combined with water in the absence of hydrofluoric acid to produce the fluoride-free mixture. Examples of suitable polar organic solvents include dimethyl sulfoxide, dimethylacetamide, dimethylformamide, and ethylene glycol. The volume ratio of solvent to water is not critical but generally the water/solvent mixture comprises at least 50 vol. %, such as at least 60 vol. %, such as at least 70 vol. % water, with the remainder the polar organic solvent.

Any water-soluble aluminum salt can be used in the present process, including, for example, aluminum chloride, bromide, iodide, fluoride, nitrate, acetate, formate and sulphate. Generally aluminum nitrate is preferred.

Similarly, any known chelated iron compound, especially Fe′ compound, can be used in the present process. In particular, it is found that the use of a chelated iron starting material, as compared with a conventional iron salt, appears to be important in allowing for better control of iron incorporation into the framework of the MOF. Suitable iron chelates include iron dionate compounds, such as iron acetylacetonoate, iron tris(2,6-dimethyl-3,5-heptanedionate), or iron tris(2,2,6,6-tetramethyl-3,5-heptanedionate). These iron chelates can be added directly or generated in situ.

The relative amounts of aluminum salt and iron chelate in the reaction mixture used in the present process will depend on the desired composition of the final MIL-53 material, but generally the reaction mixture should contain at least 10 mol. %, such as from 18 to 90 mol. %, aluminum salt, based on the total metal content of the mixture.

In addition to the aluminum salt and iron chelate, the reaction mixture used in the present process contains 1,4-benzenedicarboxylic acid or a substituted derivative or a salt thereof. Suitable 1,4-benzenedicarboxylic acid salts include sodium, potassium and ammonium salts. Suitable 1,4-benzenedicarboxylic acid derivatives include halo-substituted derivatives, such as chloro-substituted derivatives. In some embodiments, the amount of 1,4-benzenedicarboxylic acid component present in the reaction mixture varies from 50 to 300 mol %, such as from 150 to 250 mol %, of the total amount of aluminum salt and iron chelate in the reaction mixture.

Reaction between the aluminum salt, iron chelate and 1,4-benzenedicarboxylic acid in the presence of the mixed water and polar organic solvent can be conducted over a wide range of temperatures and times, with lower temperatures requiring longer times for high MOF production. In embodiments, the reaction temperature is less than 200° C., such as from 25° C. to 150° C., for example from 50° C. to 150° C., such as from 75° C. to 125° C. Reaction times are normally at least 6 hours, such as from 12 to 96 hours.

The product of the process described herein is terephthalate metal organic framework (MOF) having a flexible structure similar to or the same as that of MIL-53 and comprising iron and aluminum cations. When subjected to X-ray diffraction analysis at 200° C. under a flowing atmosphere of N₂, the resultant exhibits a pattern including at least the characteristic lines listed in Table 1:

TABLE 1

| Interplanar | Relative Intensity |
|---|---|
| d-Spacing (Å) | (100 × I/Io) |
| 10.0 ± 0.2 | s-vs |
| 6.5 ± 0.2 | w-s |
| 4.9 ± 0.2 | w-m |
| 3.34 ± 0.2 | w |

All X-ray diffraction data reported herein were collected with a Panalytical X'Pert Pro diffraction system with an Xcelerator multichannel detector, equipped with a germanium solid state detector, using copper K-alpha radiation, and on an Anton Paar HTK600 sample stage set to 200° C. under a flowing atmosphere of N₂. The diffraction data were recorded by step-scanning at 0.02 degrees of two-theta, where theta is the Bragg angle, and using an effective counting time of 2 seconds for each step. The interplanar spacings, d-spacings, were calculated in Angstrom units, and the relative intensities of the lines, I/I₀ is the ratio of the peak intensity to that of the intensity of the strongest line, above background. The intensities are uncorrected for Lorentz and polarization effects. The relative intensities are given in terms of the symbols vs=very strong (75-100), s=strong 50-74), m=medium (25-49) and w=weak (0-24).

In particular, it is found that, as the aluminum content of the final material is increased, the X-ray powder diffraction pattern indicates that the presence of the large pore form of MIL-53 increases. As is discussed in the following Examples, this is particularly evident from the variation in intensity and position of the X-ray lines centered at d-spacing values of 6.5 Å and 10 Å.

The product of the process described herein may be further characterized by methane adsorption in that the product exhibits an inflection in the gravimetric methane adsorption isotherm at a methane pressure below 8 bar (the pressure of the inflection in the methane adsorption isotherm for pure MIL-53 (Fe)) and typically at methane pressures of 6 bar or less. In some embodiments, the MOF product, when subjected to methane adsorption measurements at 30° C. displays exhibits an adsorption capacity at a methane pressure of 20 bar of greater than 2 mmol/g of the MOF product. Gas adsorption isotherms were conducted on a Hiden Isochema IGA gravimetric gas adsorption analyzer at 30° C.

The aluminum and iron-containing MIL-53 produced by the present process is useful in a variety of applications, including as a catalyst or as an adsorbent for small hydrocarbon molecules, particularly C₄₋ molecules, especially methane-containing mixtures, such as natural gas. As is well known, natural gas typically contains >85 mol. % methane, <10 mol. % ethane, and smaller quantities of propane and butanes.

### EMBODIMENTS

Embodiment 1. A process for producing a bimetallic, terephthalate metal organic framework (MOF) having a flexible structure and comprising aluminum and iron cations, the process comprising:

(a) providing a fluoride-free mixture of water and a polar organic solvent;

(b) contacting a water-soluble aluminum salt, a chelated iron compound and 1,4-benzenedicarboxylic acid or a derivative or a salt thereof with the mixture at a reaction temperature of less than 200° C. to produce a solid reaction product comprising the MOF; and

(c) recovering the MOF from the mixture,

wherein the recovered MOF product, when subjected to X-ray diffraction analysis at 200° C. under a flowing atmosphere of N₂, exhibits a pattern including at least the characteristic lines listed in Table 1:

TABLE 1

| Interplanar | Relative Intensity |
|---|---|
| d-Spacing (Å) | (100 × I/Io) |
| 10.0 ± 0.2 | s-vs |
| 6.5 ± 0.2 | w-s |
| 4.9 ± 0.2 | w-m |
| 3.34 ± 0.2 | w |

Embodiment 2. A process for producing a bimetallic, terephthalate metal organic framework (MOF) having a flexible structure and comprising aluminum and iron cations, the process comprising:

(a) providing a fluoride-free mixture of water and a polar organic solvent;

(b) contacting a water-soluble aluminum salt, a chelated iron compound and 1,4-benzenedicarboxylic acid or a derivative or a salt thereof with the mixture at a reaction temperature of less than 200° C. to produce a solid reaction product comprising the MOF; and

(c) recovering the MOF from the mixture,

wherein the recovered MOF product, when subjected to methane adsorption measurements at 30° C. displays an inflection in the adsorption isotherm at pressures bellow 8 bar.

Embodiment 3. The process of embodiment 2 where the MOF product, when subjected to methane adsorption measurement at 30° C., exhibits an adsorption capacity at 20 bar of methane of greater than 2 mmol/g.

Embodiment 4. The process of any one of embodiments 1 to 3, wherein the polar solvent comprises at least one of dimethyl sulfoxide, dimethylacetamide, dimethylformamide, and ethylene glycol.

Embodiment 5. The process of any one of embodiments 1 to 4, wherein the chelated iron compound comprises an iron dionate compound

Embodiment 6. The process of any one of embodiments 1 to 5, wherein the chelated iron compound comprises at least one of iron acetylacetonate, iron tris(2,6-dimethyl-3,5-heptanedionate), and/or iron tris(2,2,6,6-tetramethyl-3,5-heptanedionate).

Embodiment 7. The process of any one of embodiments 1 to 6, wherein the chelated iron compound is formed in situ during the contacting step (b).

Embodiment 8. The process of any one of embodiments 1 to 6, wherein the chelated iron compound is preformed and added to the contacting step (b).

Embodiment 9. The process of any one of embodiments 1 to 8, wherein the reaction temperature is from 25° C. to 150° C.

Embodiment 10. The process of any one of embodiments 1 to 9, wherein the contacting is conducted for a period of at least 6 hours.

Embodiment 11. The process of any one of embodiments 1 to 10, wherein the MOF recovered in (c) contains at least 10 mol. % aluminum, based on the total metal content of the MOF as measured by energy-dispersive X-ray spectroscopy (EDX).

Embodiment 12. The process of any one of embodiments 1 to 11, wherein the MOF recovered in (c) contains up to 90 mol. % aluminum, based on the total metal content of the MOF as measured by energy-dispersive X-ray spectroscopy (EDX).

Embodiment 13. A metal organic framework (MOF) having the structure of MIL-53 and comprising iron and aluminum cations produced by the process of any one of embodiments 1 to 12.

Embodiment 14. A process for adsorbing a gas comprising at least one C₄₋ hydrocarbon, the process comprising contacting the gas with the MOF of embodiment 13.

The invention will now be more particularly described with reference to the following non-limiting Examples and the accompanying drawings.

### Example 1: Synthesis of MIL-53 (Fe54/A146)

82 mg of terephthalic acid, 244 mg of iron (III) acetylacetonoate, and 112 mg of aluminum nitrate nonahydrate were dissolved in 10 mL of a 20% (v/v) solution of dimethyl sulfoxide in water. This solution was heated with magnetic stirring for 3 days at 120° C. After cooling to room temperature, the solids were isolated via centrifugation. These solids were washed with water (10 mL×2) followed by dimethylformamide (10 mL×1). The solids were then suspended in dimethylformamide at 100° C. overnight to remove any soluble impurities. The solids were then recollected and washed with methanol (10 mL×3) and dried at 70° C. The X-ray diffraction pattern of the resultant product at 200° C. under a flowing atmosphere of N₂ is shown in Table 2 below and suggests the product is a mixture of the large pore and narrow pore phases of MIL-53(A1), with some lines shifted probably due to the presence of iron.

TABLE 2

| Interplanar | | Relative Intensity |
|---|---|---|
| d-Spacing (Å) | Two-theta | (100 × I/Io) |
| 9.9633 | 8.868 | 100 |
| 6.5626 | 13.481 | 0.9 |
| 5.8363 | 15.169 | 6.85 |
| 5.0516 | 17.542 | 13.9 |
| 4.7893 | 18.511 | 2 |
| 4.4180 | 20.082 | 2.2 |
| 3.5411 | 25.128 | 1.2 |
| 3.3714 | 26.415 | 2.5 |
| 2.7681 | 32.314 | 0.1 |
| 2.7122 | 33.000 | 1.6 |
| 2.5296 | 35.457 | 1.3 |

Estimation of the metal content of the resulting MOF material was made by energy-dispersive X-ray spectroscopy (EDX) on a Hitachi 4800 HR-SEM using a ThermoFisher Scientific UltraDry EDS Detector. The test showed that the iron and aluminum contents of the MOF product were consistent with those of the reaction mixture (namely with a Fe/Al molar ratio of about 50:50), although EDX measurements are inherently semi-quantitative and the values obtained can have an error range of ±20%.

### Example 2: Synthesis of MIL-53 (Fe30/A170)

The process of Example 1 was repeated but with the amounts of iron (III) acetylacetonoate and aluminum nitrate nonahydrate adjusted to 104 mg and 262 mg respectively. The X-ray diffraction pattern of the resultant product at 200° C. under a flowing atmosphere of N₂ is shown in Table 3 below and again suggests the product is a mixture of the large pore/narrow pore phases of MIL-53(A1), with some lines shifted probably due to the presence of iron.

TABLE 3

| Interplanar | | Relative Intensity |
|---|---|---|
| d-Spacing (Å) | Two-theta | (100 × I/Io) |
| 9.9444 | 8.885 | 100 |
| 6.6606 | 13.282 | 2.8 |
| 5.8169 | 15.219 | 8.4 |
| 4.9999 | 17.725 | 14.9 |
| 4.3857 | 20.232 | 1.7 |
| 3.5176 | 25.298 | 1.0 |
| 3.3427 | 26.646 | 1.8 |
| 2.7581 | 32.434 | 0.3 |
| 2.7068 | 33.068 | 1.5 |

Again EDX measurements indicated that the Fe/Al molar ratio of the MOF product was consistent with that of the starting mixture.

### Example 3: Synthesis of MIL-53 (Fe83/A117)

The process of Example 1 was repeated but with the amounts of iron (III) acetylacetonoate and aluminum nitrate nonahydrate adjusted to 174 mg and 186 mg respectively. The X-ray diffraction pattern of the resultant product at 200° C. under a flowing atmosphere of N₂ is shown in Table 4 below and again suggests the product is a mixture of the large pore/narrow pore phases of MIL-53(A1), with some lines shifted and intensities changed probably due to the presence of iron.

TABLE 4

| Interplanar | | Relative Intensity |
|---|---|---|
| d-Spacing (Å) | Two-theta | (100 × I/Io) |
| 10.0943 | 8.753 | 63.3 |
| 6.6366 | 13.330 | 100 |
| 4.8544 | 18.261 | 36.8 |
| 3.3387 | 26.678 | 6.3 |
| 2.6845 | 33.350 | 18.1 |
| 2.5010 | 35.877 | 8.6 |

Again EDX measurements indicated that the Fe/Al molar ratio of the MOF product was consistent with that of the starting mixture.

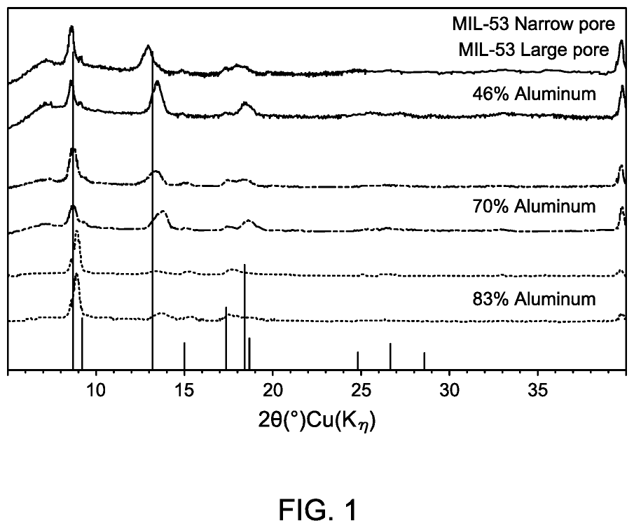

FIG. 1 shows the results of variable temperature X-ray diffraction analysis of the products of Examples 1 to 3, with patterns being taken at 30° C. and 200° C. It will be seen that as more aluminum is present in the final material, the powder diffraction pattern begins to take on a more “large pore” character. This is evident by the decrease in the relative intensity of the peak centered at 13.5° 20 as well as at that centered at 17.5° 20. Additionally, FIG. 1 shows that when these materials are heated to 200° C.; the “narrow pore” features diminish. This is particularly evident by observing the relative intensity of the peak centered at 9° 20, as well as that at 17.5° 20. This data indicates that the 1-dimenstional structure characteristic of MIL-53 materials is intact in the products from all three synthesis conditions.

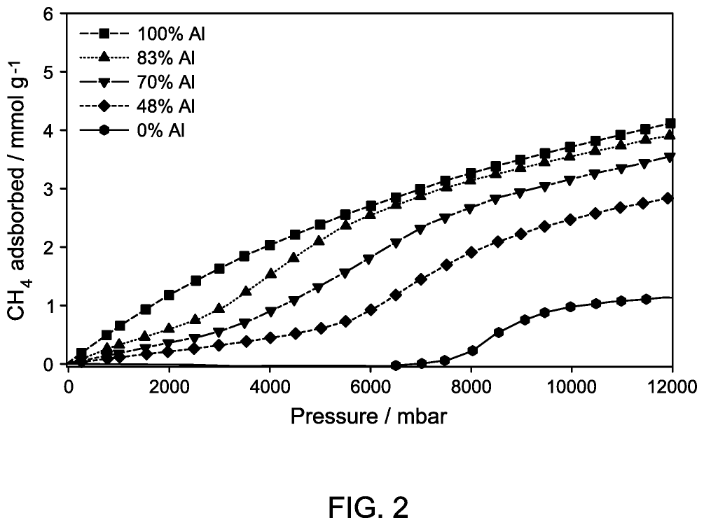

FIG. 2 compares the gravimetric methane adsorption isotherms conducted at 30° C. on the products of Examples 1 to 3 with those of MIL-53 samples containing 100% aluminum and 100% iron. It will be seen that the 100% aluminum and 100% iron MIL-53 materials exhibit classic type I and type V isotherms respectively. The isotherms for the mixed-metal materials of Examples 1 to 3 demonstrate that the pressure at which the material “opens” to the large pore form can be shifted by varying the Al/Fe ratio. Additionally, the materials produced by the present process have the desired property of opening into the “large pore” phase as opposed to some intermediary phase.

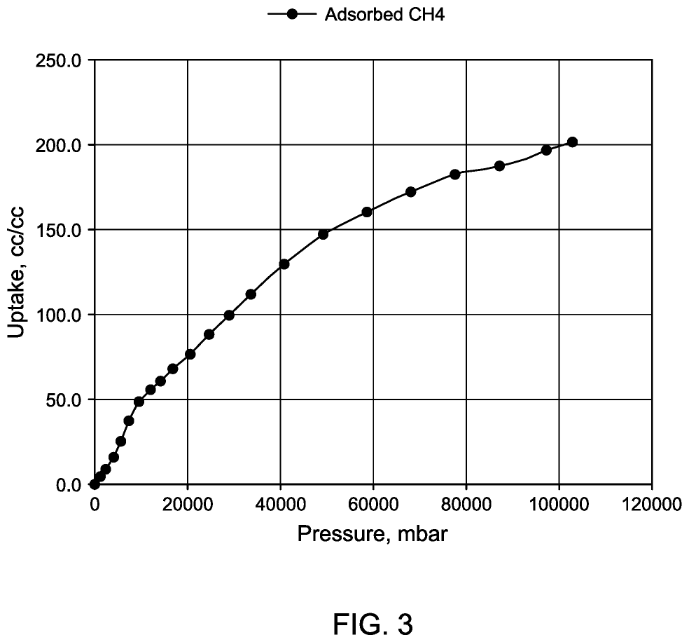

FIG. 3 shows the volumetric methane adsorption isotherm conducted at 30° C. of the product of Example 1. It will be seen that this MOF specific composition goes through a two-step process of pore opening (between 0-5 bar and 10-40 bar). Each phase change is endothermic. The endothermic phase change compensates for the heat of adsorption, an important attribute for methane storage applications.

While the present invention has been described and illustrated by reference to particular embodiments, those of ordinary skill in the art will appreciate that the invention lends itself to variations not necessarily illustrated herein. For this reason, then, reference should be made solely to the appended claims for purposes of determining the true scope of the present invention.

1. A process for producing a bimetallic, terephthalate metal organic framework (MOF) having a flexible structure and comprising aluminum and iron cations, the process comprising:
(a) providing a fluoride-free mixture of water and a polar organic solvent;
(b) contacting a water-soluble aluminum salt, a chelated iron compound and a 1,4-benzenedicarboxylic acid or a derivative or a salt thereof with the mixture at a reaction temperature of less than 200° C. to produce a solid reaction product comprising the MOF; and
(c) recovering the MOF from the mixture,
wherein the recovered MOF product, when subjected to X-ray diffraction analysis at 200° C. under a flowing atmosphere of N₂, exhibits a pattern including at least the characteristic lines listed in Table 1:
TABLE 1

| Interplanar | Relative Intensity |
|---|---|
| d-Spacing (Å) | (100 × I/Io) |
| 10.0 + 0.2 | s-vs |
| 6.5 ± 0.2 | w-s |
| 4.9 ± 0.2 | w-m |
| 3.34 ± 0.2 | w |

2. The process of claim 1, wherein the polar solvent comprises at least one of dimethyl sulfoxide, dimethylacetamide, dimethylformamide, and ethylene glycol.

3. The process of claim 1, wherein the chelated iron compound comprises an iron dionate compound

4. The process of claim 1, wherein the chelated iron compound comprises at least one of iron acetylacetonate, iron tris(2,6-dimethyl-3,5-heptanedionate), and/or iron tris(2,2,6,6-tetramethyl-3,5-heptanedionate).

5. The process of claim 1, wherein the chelated iron compound is formed in situ during the contacting step (b).

6. The process of claim 1, wherein the chelated iron compound is preformed and added to the contacting step (b).

7. The process of claim 1, wherein the reaction temperature is from 25° C. to 150° C.

8. The process of claim 1, wherein the contacting is conducted for a period of at least 6 hours.

9. The process of claim 1, wherein the MOF recovered in (c) contains at least 10 mol. % aluminum, based on the total metal content of the MOF as measured by energy-dispersive X-ray spectroscopy (EDX).

10. The process of claim 1, wherein the MOF recovered in (c) contains up to 90 mol. % aluminum, based on the total metal content of the MOF as measured by energy-dispersive X-ray spectroscopy (EDX).

11. A process for producing a bimetallic, terephthalate metal organic framework (MOF) having a flexible structure and comprising aluminum and iron cations, the process comprising:
(a) providing a fluoride-free mixture of water and a polar organic solvent;
(b) contacting a water-soluble aluminum salt, a chelated iron compound and 1,4-benzenedicarboxylic acid or a derivative or a salt thereof with the mixture at a reaction temperature of less than 200° C. to produce a solid reaction product comprising the MOF; and
(c) recovering the MOF from the mixture,
wherein the recovered MOF product, when subjected to methane adsorption measurements at 30° C. displays an inflection in the methane adsorption isotherm at a pressure below 8 bar.

12. The process of claim 11 where the MOF product, when subjected to methane adsorption measurement at 30° C., exhibits an adsorption capacity at 20 bar of methane of greater than 2 mmol/g.

13. The process of claim 11, wherein the polar solvent comprises at least one of dimethyl sulfoxide, dimethylacetamide, dimethylformamide, and ethylene glycol.

14. The process of claim 11, wherein the chelated iron compound comprises an iron dionate compound

15. The process of claim 11, wherein the chelated iron compound comprises at least one of iron acetylacetonate, iron tris(2,6-dimethyl-3,5-heptanedionate), and/or iron tris(2,2,6,6-tetramethyl-3,5-heptanedionate).

16. The process of claim 11, wherein the chelated iron compound is formed in situ during the contacting step (b).

17. The process of claim 11, wherein the chelated iron compound is preformed and added to the contacting step (b).

18. The process of claim 11, wherein the reaction temperature is from 25° C. to 150° C.

19. The process of claim 11, wherein the contacting is conducted for a period of at least 6 hours.

20. The process of claim 11, wherein the MOF recovered in (c) contains at least 10 mol. % aluminum, based on the total metal content of the MOF as measured by energy-dispersive X-ray spectroscopy (EDX).

21. The process of claim 11, wherein the MOF recovered in (c) contains up to 90 mol. % aluminum, based on the total metal content of the MOF as measured by energy-dispersive X-ray spectroscopy (EDX).

22. A metal organic framework (MOF) having the structure of MIL-53 and comprising iron and aluminum cations produced by the process of claim 1.

23. A process for adsorbing a gas comprising at least one C₄₋ hydrocarbon, the process comprising contacting the gas with the MOF of claim 22.

24. A metal organic framework (MOF) having the structure of MIL-53 and comprising iron and aluminum cations produced by the process of claim 11.

25. A process for adsorbing a gas comprising at least one C₄₋ hydrocarbon, the process comprising contacting the gas with the MOF of claim 24.
